(19) United States
(12) Patent Application Publication
(10) Pub. No.: US 2009/0234334 A1
(43) Pub. Date: Sep. 17, 2009

(54) Spatially Distributed Spectrally Neutral Optical Attenuator

(76) Inventors: Alexander Artsyukhovich, Dana Point, CA (US); T. Scott Rowe, Dana Point, CA (US); Bruno Lassalas, Irvine, CA (US); Lawrence Darnell, Cypress, TX (US); W. Jake Kozlowski,, JR., Tega Cay, SC (US)

Correspondence Address: ALCON, IP LEGAL, TB4-8, 6201 SOUTH FREEWAY, FORT WORTH, TX, 76134, US

(21) Appl. No.: 12/410,115

(22) Filed: Mar. 24, 2009

Related U.S. Application Data
(63) Continuation of application No. 11/204,305, filed on Aug. 15, 2005, now abandoned.
(60) Provisional application No. 60/601,364, filed on Aug. 13, 2004.

(51) Int. Cl. A61F 9/007; A61B 18/18

(52) U.S. Cl. 606/4

(57) ABSTRACT

A system, apparatus and method for spatially distributed, spectrally neutral optical attenuation. One embodiment of the apparatus can include: an attenuator fin plate; a set of attenuator fins, wherein each of the fins is operably coupled to the fin plate at a preset fin angle to the fin plate normal such that the attenuator fins maintain their position relative to the fin plate as the fin plate moves; and a motor for rotating the fin plate a set angular distance around an axis of rotation, wherein the axis of rotation is at a preset fin plate angle to a light beam direction of travel and wherein the attenuator fins block varying amounts of the light beam as the fin plate is rotated through the set angular distance. The attenuator fin plate and attenuator fins can be a single, integral component, wherein the attenuator fin plate is etched and stamped to form the attenuator fins, or separately formed components that are attached, for example, to a separate frame. The embodiments of the attenuator of this invention can be configured for use within an ophthalmic high brightness illumination system.

### TECHNICAL FIELD OF THE INVENTION

This application claims priority from U.S. patent application Ser. No. 60/601,364 filed Aug. 13, 2004. This application also claims priority from, and is a continuation of U.S. patent application Ser. No. 11/204,305 filed on Aug. 15, 2005.

The present invention relates generally to surgical instrumentation. In particular, the present invention relates to surgical instruments for illuminating a surgical area during eye surgery. Even more particularly, the present invention relates to spatially distributed, spectrally neutral optical attenuators for such surgical instruments that can homogeneously attenuate an optical beam.

### BACKGROUND OF THE INVENTION

Many ophthalmic surgical procedures performed on a patient's eye require illuminating a portion of the eye so that a surgeon can properly observe the surgical site. In ophthalmic surgery, various different types of instruments are known and available for use by a surgeon to illuminate the interior of the eye. The handheld (probe) portion of a typical ophthalmic illuminator comprises a handle having a projecting tip and a length of optical fiber that enters a proximal end of the handle and passes through the handle and the tip to a distal end of the tip, from which light traveling along the optical fiber can project. The proximal end of the optical fiber can be optically coupled to a light source, such as in a high brightness illuminator, as known to those having skill in the art, to provide the light that is transmitted through the fiber. This type of handheld illuminator is typically used by inserting the probe tip through a small incision in the eye. In this way, light from the illuminator light source is carried along the optical fiber though the handpiece and emitted from the distal end of the probe to illuminate the surgical site for the surgeon. Ophthalmic illuminators that use a length of optical fiber to direct light from the light source to a surgical site are well known in the art.

A typical ophthalmic illumination system comprises the handheld portion, or probe, to deliver illumination from a light source housed in an enclosure. Along with the light source, the enclosure typically houses optics that guide light from the light source to the optical fiber of the probe, a power supply, electronics with signal processing, and associated connectors, displays and other interfaces as known in the art. In addition, a typical ophthalmic illumination system includes an attenuator. Attenuators are used to vary the intensity of an optical beam to control the intensity profile of the light spot (focal spot) provided by the optical beam. Attenuating an optical beam provides the surgeon a means to attain a desired light intensity at a surgical site. Further, beam attenuation is typically configured such that the surgeon can vary the degree of attenuation as needed.

Optical attenuators are known in the art for attenuating a collimated beam from an ophthalmic illumination system's light source. Some prior art optical attenuators have a design that changes the degree of attenuation across the cross-section of the light beam. For example, the attenuator disclosed in U.S. Pat. No. 4,425,599, issued to Volpi, performs in this manner. This non-homogeneous attenuation across the cross-section of the light beam, however, is not desirable in many applications, in particular in systems that focus light into multimode fibers, as it can result in different modes (angles) being attenuated in a non-equal manner. This can result in ring structures appearing in the beam output focal spot.

Some large scale prior art attenuators are exemplified by the well-known Venetian blind and marine signal lights, such as the well-known naval signal lamps in use by navies around the world. Both these systems use rotating plates to block a light beam. These systems, however, use individually rotating fins (blinds) to attenuate the light beam. Other prior art small scale attenuators used in ophthalmic illumination systems are disclosed in U.S. Pat. Nos. 6,404,970 to Gransden et al., and 6,367,958 and 5,006,965 to E. M. Jones. These prior art attenuators, however, do not provide color neutrality, compact size, a direct motor drive or homogeneous attenuation across the entire light aperture.

Therefore, a need exists for an optical attenuator for use within an ophthalmic illumination system that can provide for variable attenuation of wide aperture optical (e.g., UV, Visible, IR) beams in a homogeneous manner over the entire aperture. Further still, a need exists for such an attenuator that can provide spectrally neutral attenuation within the desired range of attenuation.

### BRIEF SUMMARY OF THE INVENTION

The embodiments of the system, apparatus and method for spatially distributed, spectrally neutral optical attenuation of the present invention substantially meet these needs and others. One embodiment of the apparatus of the present invention is an attenuator for attenuating a light beam, comprising: an attenuator fin plate; a set of attenuator fins, wherein each of the fins is operably coupled to the fin plate at a preset fin angle to the fin plate normal such that the attenuator fins maintain their position relative to the fin plate as the fin plate moves; and a means for rotating the fin plate a set angular distance around an axis of rotation, wherein the axis of rotation is at a preset fin plate angle to the light beam direction of travel and wherein the attenuator fins block varying amounts of the optical beam as the fin plate is rotated through the set angular distance.

The attenuator fin plate and attenuator fins can be a single, integral component, wherein the attenuator fin plate is etched and stamped to form the attenuator fins, or separately formed components that are attached, for example, to a separate frame. The means for rotating the fin plate would then comprise means to rotate the attenuator frame. Means for rotating the attenuator fin plate or frame can include a stepper motor, for discrete step positions, or a continuously variable motor for infinitely variable positioning. The means for rotating the attenuator fin plate or frame can be electronically controlled, for example, by a microprocessor on a printed circuit board or other such controller as known to those having skill in the art.

The preset fin angle can be 31 degrees, and the preset fin plate angle can be 90 degrees. Each of the attenuator fins can be operably coupled to the fin plate at the same preset fin angle and the fin plate and/or frame centered on the axis of rotation. Each fin's major axis can be parallel to every other fin's major axis, and the axis of rotation can be parallel to each fin's major axis. The set of attenuator fins can comprise eight attenuator fins and the attenuator fins can be spaced equally apart from one another. The attenuator fin plate and set of attenuator fins can be sized so as to interfere with the entire light beam cross-section/aperture at a position along the set angular distance corresponding to zero percent of the optical beam passing through the attenuator fins. The embodiments of the attenuator of this invention can be configured for use within an ophthalmic high brightness illumination system.

Other embodiments of the present invention can include a system and a method for spatially distributed, spectrally neutral optical attenuation of an optical beam using an optical attenuator in accordance with the teachings disclosed herein.

Embodiments of this invention can be implemented within a surgical machine or system for use in ophthalmic or other surgery. In particular, it is contemplated that the system, apparatus and method for spatially distributed, spectrally neutral optical attenuation of this invention can be implemented or incorporated into any ophthalmic illumination system in which it is desirable to attenuate an optical beam in a homogeneous and spatially neutral manner. Other uses for the embodiments of this invention will be apparent to those having skill in the art.

### BRIEF DESCRIPTION OF THE SEVERAL VIEWS OF THE DRAWINGS

A more complete understanding of the present invention and the advantages thereof may be acquired by referring to the following description, taken in conjunction with the accompanying drawings, in which like reference numbers indicate like features and wherein:

FIG. 1 is a simplified block diagram illustrating an exemplary high-brightness illuminator system 10 comprising an embodiment of the present invention;

FIG. 2 is a simplified block diagram illustrating in greater detail an embodiment of an optical attenuator according to the present invention;

FIG. 3 is a simplified drawing of an exemplary stamping tool for shaping a fin-plate of an embodiment of an optical attenuator of this invention;

FIGS. 4 and 5 show a MathCAD plot of two dependences used to calculate the fin angle on an embodiment of the attenuator of the present invention; and

FIG. 6 illustrates another embodiment of an attenuator of the present invention.

### DETAILED DESCRIPTION OF THE INVENTION

Preferred embodiments of the present invention are illustrated in the FIGURES, like numerals being used to refer to like and corresponding parts of the various drawings.

The various embodiments of the present invention provide for spatially distributed, spectrally neutral optical attenuation of an optical beam. In ophthalmic surgery there is often a need to attenuate a wide aperture optical beam (e.g., ultra-violet, Visible, Infra red, etc.) in a homogeneous manner (equal attenuation per square area) over the beam aperture. Further, there is a need to be able to vary the degree of attenuation as needed and to have the attenuation be spectrally neutral within the desired range of attenuation. The embodiments of the apparatus, method and system for spatially distributed, spectrally neutral optical attenuation of this invention can provide these functions by following simple principles analogous to those of Venetian blinds mounted on a solid frame.

Rotating a frame changes an angle between the frame fins and an optical beam passing between the fins. This varies the attenuation from a maximum transmission of the optical beam, which can be as high as 95% or more, to complete or near complete blockage of the beam. One purpose of the present invention is to provide ophthalmic illumination systems implementing an embodiment of this invention a means to attenuate an optical beam in a spatially distributed, spectrally neutral manner and to provide the surgeon a means to variably control the desired illumination scheme at a surgical site.

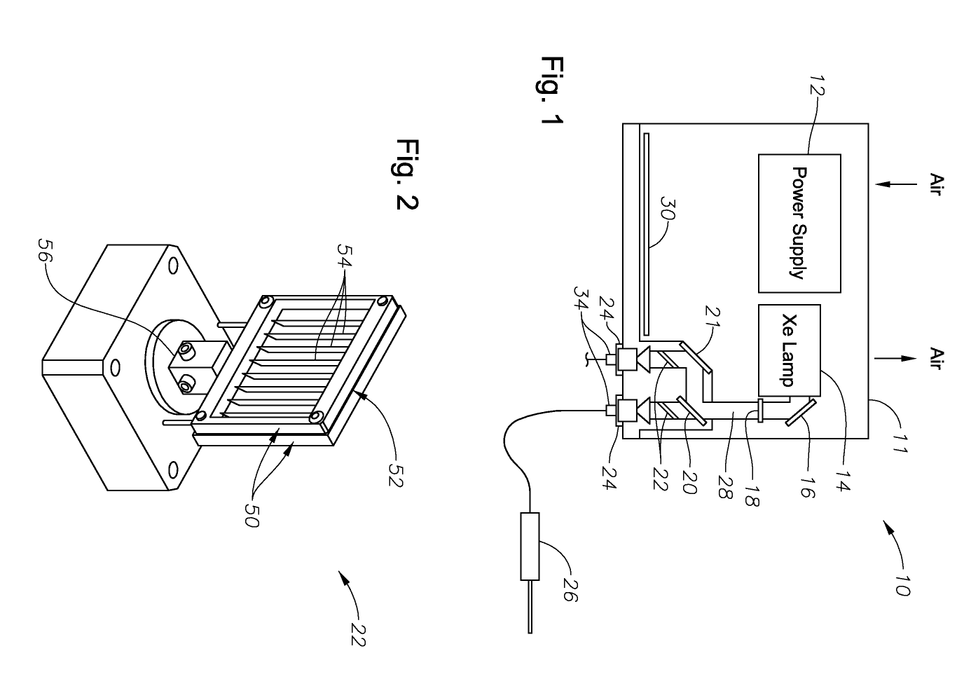

FIG. 1 is a simplified block diagram of a high brightness ophthalmic illumination system incorporating an embodiment of a spatially distributed, spectrally neutral attenuator of the present invention. Illuminator system 10 comprises power supply 12 and illumination source 14, cold mirror 16, a hot mirror 18, a beam splitter 20, mirror 21, optical fiber ports 24 and attenuators 22. Illuminator system 10 also typically comprises one or more optical fiber probes 26. Optical fiber probes 26 comprise the handheld portion of the illuminator system 10, including optical fiber 34, which is optically coupled to the illumination source 14 within enclosure 11. High brightness illuminator system 10 is exemplary only and is not intended to limit the scope of the present invention in any way. The embodiments of the present invention can be used in any such ophthalmic illuminator, medical laser, or any other system or machine in which it is desirable to attenuate an optical beam in a homogeneous and spectrally neutral manner.

Optical source 14 of illuminator system 10 in this example comprises a xenon lamp, but it can comprise any suitable light source as known to those having skill in the art. Xenon lamp 14 emits light beam 28, which is directed along the optical path comprising cold mirror 16, hot mirror 18, beam splitter 20, mirror 21, attenuators 22, and optical fiber ports 24. In this example, beam splitter 20 splits light beam 28 into two optical paths to provide for two optical probes 26 if desired. Cold mirror 16 and hot mirror 18 combine to remove the infrared components of light beam 28 (heat) and provide a cool visible light beam 28 to the downstream optical components, as will be familiar to those skilled in the art. Attenuators 22 attenuate optical beam 28 in the manner disclosed herein. Attenuators 22 can each be custom designed for its respective optical path and need not be identical, though they can be. Further, each attenuator 22 can be independently controlled via, for example, PCB 30. Although high brightness illuminator system 10 is shown comprising two optical fiber ports 24, it will be obvious to those having skill in the art single optical port 24 or multiple optical ports 24 can be implemented within illuminator system 10. Illuminator system 10 further comprises a printed circuit board (“PCB”) 30, or its electronic equivalent, to provide signal processing and control functions. PCB 30 can be implemented in any manner and configuration capable of performing the desired processing and control functions described herein, as will be apparent to those having skill in the art. Optical ports 24 comprise a receptacle to receive the proximal end of the fiber corresponding to fiber probes 26, which are inserted into the high brightness illuminator enclosure 11 and optically coupled to illumination source 14 to direct light onto a desired site.

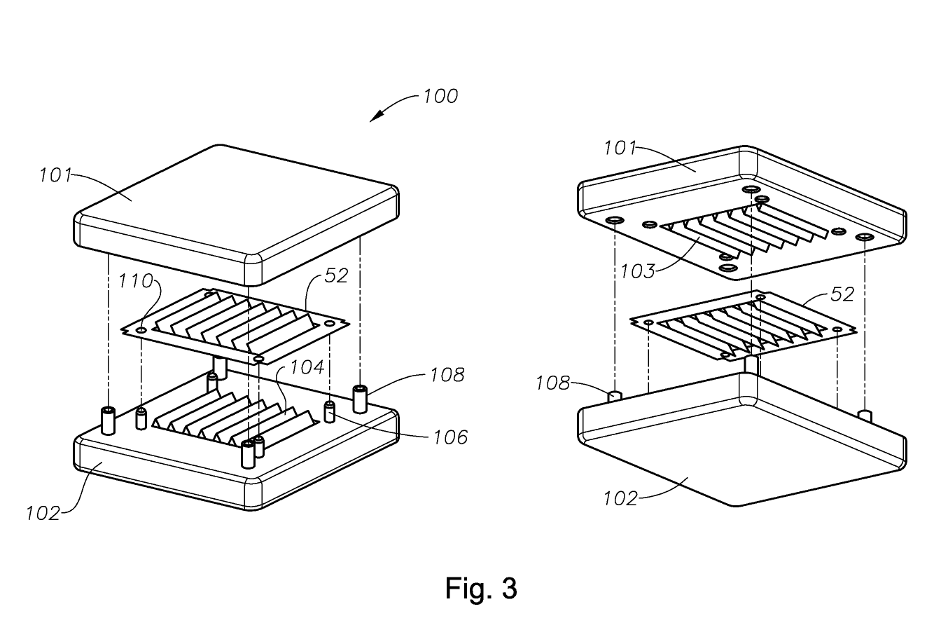

FIG. 2 is a simplified block diagram illustrating in more detail an attenuator 22 of FIG. 1. Attenuator 22 comprises an attenuator frame 50 to which is attached an attenuator fin-plate 52. Attenuator fin-plate 52 comprises fins 54 that are tilted at a preset angle to the attenuator fin-plate 52 normal. Attenuator frame 50 and fin-plate 52 can be driven by a means for rotating fin-plate 52 and/or frame 50, such as a motor 56, such that they can be rotated through a range of angles and stopped at a desired angle. As the attenuator fin-plate 52 and fins 54 are rotated, attenuation (transmission) of the light beam 28 can range between a preset maximum to a preset minimum, e.g., 95% transmission to 0% transmission. Motor 56 can be any suitable stepper motor, for discrete step positions, or a continuously variable motor for infinitely variable positioning, as will be known to those having skill in the art. Motor 56 can be electronically controlled, for example, by a microprocessor on PCB 30, or by another such controller as known to those having skill in the art.

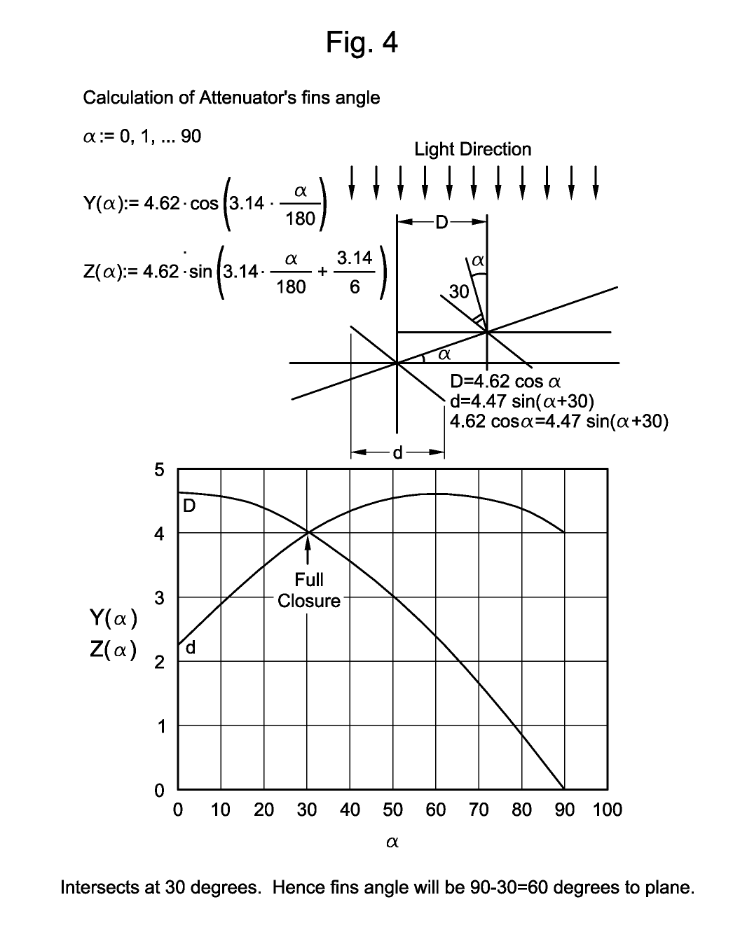

In one embodiment of the present invention, attenuator fin-plate 52 and fins 54 of attenuator 22 are made by first photo-etching a flat pattern onto an initially flat attenuator fin-plate 52 comprising copper beryllium plate. Copper beryllium alloys are known for their good shape memory, even at elevated temperatures, and are a suitable material for attenuator 22's attenuator fin-plate 52. Initially flat attenuator fin-plate 52 is then plated with bright tin coat and stamped with a tool designed for this purpose. An exemplary stamping tool 100 is shown in FIG. 3. Stamping tool 100 comprises an upper plate 101 and lower plate 102 having respective fin forms 103 and 104 tilted at an angle to the attenuator normal operable to shape fins 54 of fin-plate 52 to a desired angle (in this example, 31°). The two stamping tool 100 plates 101 and 102 are brought together forcibly in a manner that will be known to those skilled in the art to produce fins 54 on attenuator fin-plate 52 having a tilt from normal corresponding to the angle of stamping tool 100 fin forms 103 and 104. This method of manufacture is well-known in the art and stamping tool 100 can be produced by any tool forming technology as known to those skilled in art. Stamping tool 100 includes appropriate pin forms 106 and guiding pins 108 to produce fastener holes 110 in attenuator fin-plate 52 and to guide the separate portions of stamping tool 100 into position with one another.

Although the exemplary embodiment of attenuator 22, and in particular attenuator fin-plate 52 shown in FIGS. 2 and 3. have been described with reference to a particular manufacturing technique and material (e.g., copper beryllium plate), it is contemplated to be within the scope of this invention and will be familiar to those having skill in the art that attenuator 22 and attenuator fin-plate 52 can be manufactured using any appropriate material, stepper motor, and fin angle, or any combination thereof suitable to meet the requirements of a particular attenuator 22 implementation. In particular, various fin angles are contemplated for the embodiments of the present invention to achieve varying degrees of attenuation.

The fins 54 of an attenuator 22 of the present invention should be thin enough so that when the fins 54 are aligned along the collimated optical beam, such as light beam 28, the relative cross-section taken by the fins should be small. In the embodiment shown in FIGS. 2 and 3, maximum transmission achieved with an eight fin design is approximately 95% of the optical beam intensity. Minimum transmission is 0%, (i.e. the beam is blocked entirely).

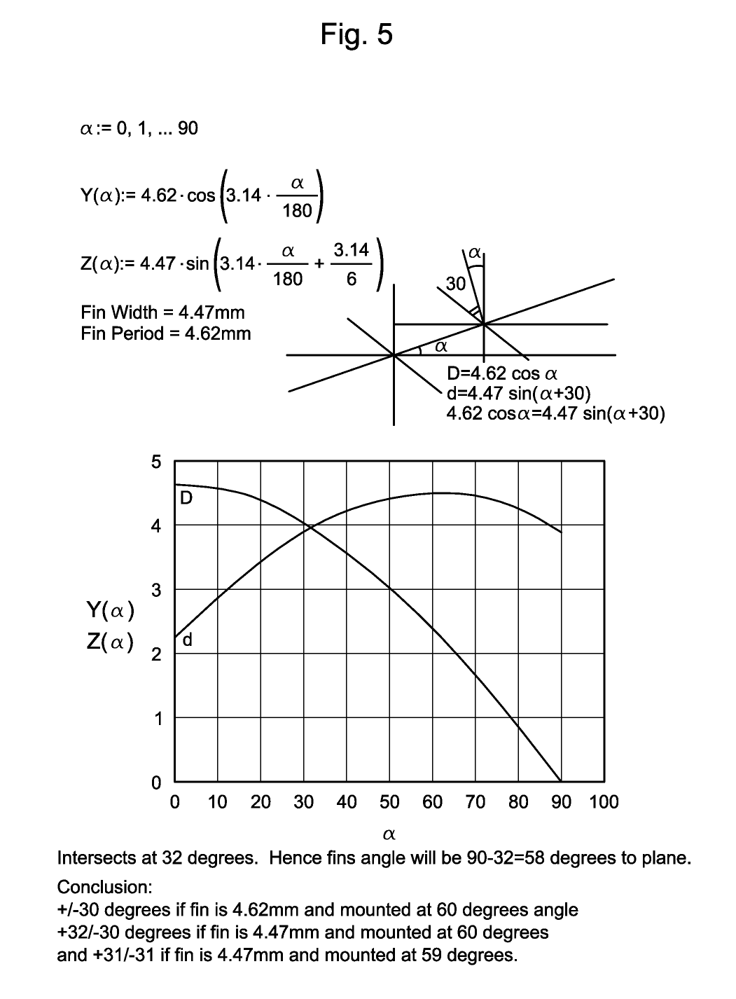

Another consideration is to select an angle (αₘₐₓ) of maximum attenuator rotation and the period of the fins 54 in such a way that a relatively small angle of rotation results in a considerable attenuation change in the light beam 28. FIGS. 4 and 5 show a MathCAD plot of two dependences—projection of fin period on optical beam cross-section as a function of attenuator tilt D(α); and, projection of the fin period on cross-section of the optical beam 28 as a function of angle of rotation d(α). At the angle that achieves D(α)=d(α), the attenuator 22 is blocking light beam 28. Calculations in FIGS. 4 and 5 are shown for fins 54 rotated by 30° from the attenuator fin-plate 52 normal. Other fin 54 angle and maximum angle of rotation combinations may be suitable for different applications and are contemplated to be within the scope of the present invention.

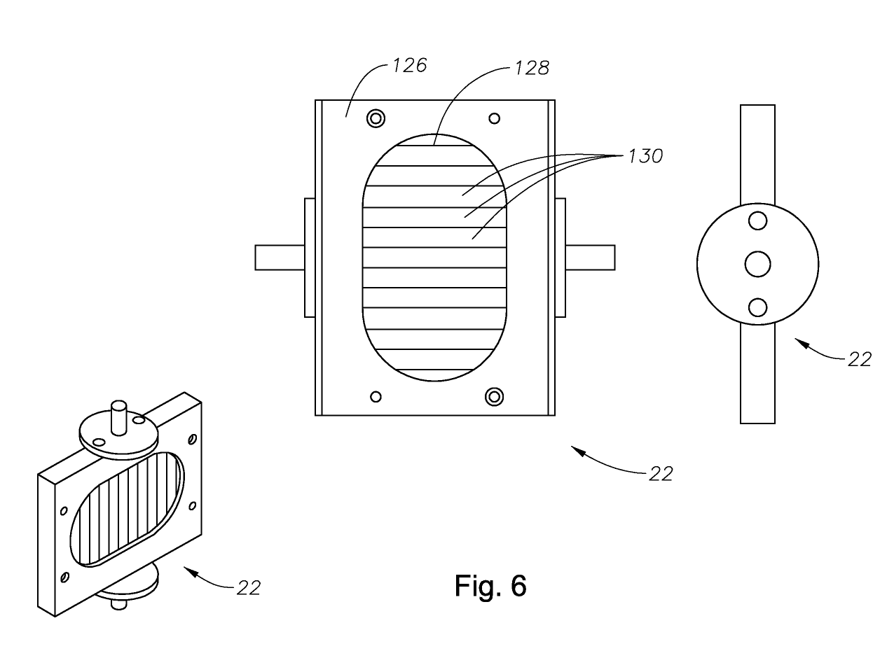

FIG. 6 shows another embodiment of an attenuator 22 in accordance with the present invention. Attenuator mounting plate 126 of this embodiment comprises an enclosure attached to and housing fin-plate 128. Attenuator mounting plate 126 comprises an enclosure having an elliptical in this example, although the shape can be arbitrarily selected) opening through which fins 130 of attenuator fin-plate 128 will receive and attenuate an optical beam 28. Attenuator mounting plate 126 can be operably connected to and driven by a stepper motor, such as stepper motor 56 of FIG. 2. Various other such embodiments of a mounting plate 126 and attenuator fin-plate 128 combinations are contemplated to be within the scope of this invention. For example, attenuator fin-plate 52 and attenuator fins 54 can be a single, integral component, as described above, or separately formed components. In either case they can be attached to a frame, such as frame 50, or stand alone.

In contrast to the prior art, the various embodiments of the attenuator 22 of this invention can provide homogeneous attenuation of the light beam 28, which is important in, for example, systems that focus light into multi-mode fibers. This is because different modes (angles) can be attenuated equally. Thus, no ring structures appear in the output of the optical beam upon attenuation. Periodicity of the attenuator fins is used to control how fine (how homogeneous) the attenuation will be. For the purposes of an ophthalmic illuminator, an embodiment such as the eight fin embodiment of FIG. 2 is sufficient to provide homogeneous attenuation, although a greater or lesser number of fins can be used depending on the application.

Embodiments of the apparatus, method and system for spatially distributed, spectrally neutral optical attention of the present invention provide an attenuator 22 in which the fins are mounted on a rotatable frame as opposed to some prior art attenuators in which individual fins (blinds) are rotated. Rotating the frame changes the angle between all the fins and the collimated optical beam 28 passing between them. This action can be used to vary the amount of attenuation from a maximum transmission to potentially complete blockage of the optical beam.

The various embodiments of the present invention provide various advantages, including homogeneous attenuation of an optical beam across the entire light aperture, color neutrality, a compact design and direct motor drive. Other advantages of the present invention include the ability to include a right angle between the attenuated light beam and the axis of the attenuator location (allows positioning of the attenuator motor close to the optical beam). Further, in an ophthalmic illumination system incorporating multiple attenuators 22 in accordance with the embodiments of the present invention, individual attenuators 22 can be controlled independently of one another to provide, for example, varying amounts of attenuation along different optical paths (e.g., to different optical ports 24). This independent control can be accomplished, for example, by PCB 30 in a manner that will be known to those having skill in the art.

Although the present invention has been described in detail herein with reference to the illustrated embodiments, it should be understood that the description is by way of example only and is not to be construed in a limiting sense. It is to be further understood, therefore, that numerous changes in the details of the embodiments of this invention and additional embodiments of this invention will be apparent to, and may be made by, persons of ordinary skill in the art having reference to this description. It is contemplated that all such changes and additional embodiments are within the spirit and true scope of this invention as claimed below.

What is claimed is:

1. An attenuator for homogenously attenuating a light beam, comprising:
an attenuator fin plate;
a set of attenuator fins, wherein each of the fins is operably coupled to the fin plate at a preset fin angle to the fin plate normal such that the attenuator fins maintain their position relative to the fin plate as the fin plate moves; and
a means for rotating the fin plate a set angular distance around an axis of rotation, wherein the axis of rotation is at a preset fin plate angle to the light beam direction of travel and wherein the attenuator fins block between approximately 5% and 100% of the light beam as the fin plate is rotated through the set angular distance.

2. The attenuator of claim 1, wherein the attenuator fin plate and attenuator fins are a single component, and wherein the attenuator fin plate is etched and stamped to form the attenuator fins.

3. The attenuator of claim 2, further comprising an attenuator frame, wherein the attenuator fin plate is operably coupled to the attenuator frame, and wherein the means for rotating the fin plate comprises a means to rotate the attenuator frame.

4. The attenuator of claim 1, wherein the beam of light is an optical beam.

5. The attenuator of claim 1, wherein the preset fin angle is 31 degrees to the fin plate.

6. The attenuator of claim 1, wherein the means for rotating the fin plate is a stepper motor.

7. The attenuator of claim 1, wherein the preset fin plate axis of rotation angle to the light beam is 90 degrees.

8. The attenuator of claim 1, wherein each of the fins is operably coupled to the fin plate at the same preset fin angle.

9. The attenuator of claim 1, wherein the attenuator fin plate is centered on the axis of rotation.

10. The attenuator of claim 9, wherein each fin's major axis is parallel to every other fin's major axis, and wherein the axis of rotation is parallel to each fin's major axis.

11. The attenuator of claim 1, wherein the set of attenuator fins comprises eight attenuator fins.

12. The attenuator of claim 1, wherein the attenuator fins are spaced equally apart from one another.

13. The attenuator of claim 1, wherein the attenuator fin plate and attenuator fins are made of copper beryllium.

14. The attenuator of claim 13, wherein the copper beryllium attenuator fin plate and fins are tin coated.

15. The attenuator of claim 1, wherein the light beam is a light beam produced by a xenon light source.

16. The attenuator of claim 1, wherein the means for rotating the attenuator fin plate are electronically controlled.

17. The attenuator of claim 1, wherein rotating the attenuator fin plate through the set angular distance changes the angle between the attenuator fins and the light beam such that the amount of the optical beam passing through the attenuator fins can be varied from a maximum amount to a minimum amount.

18. The attenuator of claim 17, wherein the minimum amount is zero percent.

19. The attenuator of claim 18, wherein the attenuator fin plate and set of attenuator fins are sized so as to interfere with the entire light beam cross-section/aperture at a position along the set angular distance corresponding to zero percent of the optical beam passing through the attenuator fins.

20. The attenuator of claim 1, wherein the attenuator is configured for use within an ophthalmic high brightness illumination system.

21. An attenuator for attenuating a light beam, comprising:
an attenuator frame;
a set of attenuator fins, wherein each of the fins is operably coupled to the frame such that the fins are positioned inside the frame and aligned on a common plane defined by the frame and wherein each fin's major axis is parallel to every other's fin's major axis and to a common fin axis of rotation, wherein each fin's major axis is positioned a preset distance from a neighboring fin's major axis, and wherein each fin is at a preset angle to the frame normal such that the fins maintain their position relative to the frame as the frame moves; and
a means for rotating the frame and fins around the common fin axis of rotation, wherein the common fin axis of rotation is at a preset angle to the light beam direction of travel, and wherein the frame is centered along the common fin axis of rotation.

22. An attenuator for homogenously attenuating a beam of electro-magnetic radiation, comprising:
an attenuator fin plate;
a set of attenuator fins, wherein each of the fins is operably coupled to the fin plate at a preset fin angle to the fin plate normal such that the attenuator fins maintain their position relative to the fin plate as the fin plate moves; and
a means for rotating the fin plate a set angular distance around an axis of rotation, wherein the axis of rotation is at a preset fin plate angle to the beam of electro-magnetic radiation direction of travel and wherein the attenuator fins block between approximately 5% and 100% of the beam of electromagnetic radiation as the fin plate is rotated through the set angular distance.
